(19) United States
(12) Patent Application Publication
(10) Pub. No.: US 2025/0152639 A1
(43) Pub. Date: May 15, 2025

(54) OVARIAN CARE FORMULA CONTAINING A-SDGPLUS AND PREPARATION METHOD THEREFOR

(71) Applicant: Yangfan Lin, Hangzhou (CN)

(72) Inventor: Yangfan Lin, Hangzhou (CN)

(21) Appl. No.: 19/023,409

(22) Filed: Jan. 16, 2025

(51) Int. Cl. A61K 35/741; A61K 36/53; A61K 36/55

(52) U.S. Cl. CPC A61K 35/741; A61K 36/53; A61K 36/55; A61K 2236/17; A61K 2236/30

(57) ABSTRACT

An ovarian care formula containing A-SDGPLUS and a preparation method therefor are provided, belonging to the technical field of ovarian care. The ovarian care formula containing A-SDGPLUS is prepared from the following raw materials in percentage by mass: 30%-50% of postbiotics, 15%-25% of chasteberry extract and 5%-10% of flaxseed lignan. The preparation method for the ovarian care formula containing A-SDGPLUS includes the following steps: mixing postbiotics, a chasteberry extract and flaxseed lignan in percentage by mass described above, evenly stirring, and packaging to obtain an ovarian care solid drink. The ovarian care formula is composed of postbiotics, the chasteberry extract and flaxseed lignan, which can be directly taken to act on ovary, with quick effect and no side effects.

### TECHNICAL FIELD

The present disclosure belongs to the technical field of ovarian care, and particularly relates to an ovarian care formula containing A-SDGPLUS and a preparation method therefor.

### BACKGROUND

Ovary, as an important reproductive organ in a woman's body, plays a crucial role in a female endocrine system. The health condition of the ovary is directly related to fertility, endocrine balance and overall health of women. Therefore, ovarian care is especially important.

The current ovarian care methods generally comprise:
(1) essential oil massage and maintenance: ovarian care is assisted by promoting blood circulation and essential oil penetration, and it is difficult for this method to directly act on the ovaries, resulting in poor care effects, as the ovaries are located in a deep position in the pelvic cavity and difficultly touched by massage;
(2) food/diet therapy care: necessary nutrients are provided for a body through increase in intake of legumes, fruits and vegetables so as to assist ovarian care, so this method is relatively safe, but slow in effect;
(3) medicine care: ovarian care is assisted by using a medicine containing a hormone such as progesterone or estradiol, so this method is quick in effect, but easily has side effect if used for a long term, so as to affect health.

In view of this, an ovarian care formula containing A-SDGPLUS and a preparation method therefore are designed in order to solve the above problem.

### SUMMARY

In order to solve the problem proposed in the above background. The present disclosure provides an ovarian care formula containing A-SDGPLUS and a preparation method therefore, which have the characteristics of directly acting on ovary, quick effect and no side effects.

Another objective of the present disclosure is to provide a preparation method for an ovarian care formula containing A-SDGPLUS.

In order to achieve the above objective, the present disclosure provides the following technical solution: provided is an ovarian care formula containing A-SDGPLUS, which is prepared from the following raw materials in percentage by mass:
30%-50% of postbiotics, 15%-25% of chasteberry extract and 5%-10% of flaxseed lignan.

Provided is a preparation method for an ovarian care formula containing A-SDGPLUS, comprising the following steps:
mixing postbiotics, a chasteberry extract and flaxseed lignan in percentage by mass described above, evenly stirring, and packaging to obtain an ovarian care solid drink.

Further, a preparation method for the postbiotics comprises:
S101: activating condensed Weissman's bacteria to obtain an activating bacterial solution of condensed Weissman's bacteria;
S102: inoculating the activating bacterial solution of condensed Weissman's bacteria into a fermentation medium to be fermented to obtain a fermentation broth of condensed Weissman's bacteria; and
S103: centrifuging the fermentation broth of condensed Weissman's bacteria, and then freeze-drying separated supernatant to obtain postbiotics.

Further, in the step S102, the fermentation medium is prepared from the following raw materials in parts by mass:
1-15 parts of peptone, 15-35 parts of yeast extract, 5-10 parts of glucose, 1-15 parts of corn starch, 1-15 parts of dipotassium hydrogen phosphate, 0.1-1 part of magnesium sulfate, 0.01-0.1 part of manganese sulfate and 500-1200 parts of distilled water.

Further, in the step S102, the fermentation temperature of the activating bacterial solution of condensed Weissman's bacteria during the fermentation is 30-50° C., the speed of a table concentrator is 100-500 rpm, and the fermentation time is 15-30 h.

Further, a preparation method for the chasteberry extract comprises:
S201: taking 500-600 parts of dried chasteberry raw material, washing, smashing, boiling, and filtering floating impurities;
S202: after repeatedly boiling three times, adding 1-10 parts of carrier maltodextrin powder dusting through a spray tower, and filtering via a 100-mesh sieve to obtain the chasteberry extract.

Further, a preparation method for the flaxseed lignan comprises:
S301: smashing flaxseed fat dregs, filtering via a 10-80 meshed sieve, adding a 70-99.9% ethanol solution and alkali, performing reflux extraction, and then filtering an extraction solution;
S302: concentrating the extraction solution to a proportion of 1.05-1.1, and adjusting pH to 6.5-7.5 with an acid;
S303: loading the concentrated solution onto a macroporous adsorbent resin, performing primary elution with water once and then performing secondary elution with 20-50% ethanol solution, concentrating and drying the concentrated solution to obtain 50-70% flaxseed lignan; and
S304: dissolving the dried flaxseed lignan crude product with a fat-soluble solvent, filtering and drying a filtrate to obtain ≥98% flaxseed lignan named A-SDGPlus, i.e., flaxseed lignan rich in ovarian care components.

Further, in the step S301, an addition ratio of ethanol solution to flaxseed fat dregs is 1:5-1:25 by weight, and an addition ratio of alkali to flaxseed fat dregs is 1-4% by weight.

Further, in the step S301, the reflux extraction temperature is 70-80° C., and the extraction time is 3-4 h.

Compared with the prior art, the present disclosure has the beneficial effects:

The ovarian care formula is composed of postbiotics, a chasteberry extract and flaxseed lignan, wherein the postbiotics can promote the synthesis of human collagen synthesis, maintain an ovarian structure and support an ovarian function, prevent premature ovarian failure, regulate endocrine function and assist in maintaining normal secretion levels of estrogen, various health problems, caused by decreased estrogen levels, including menopausal symptoms and reproductive system aging, are prevented by maintaining stable water solubility of estrogen, while enhancing the elasticity and contractility of the pelvic fascia, urethral and bladder ligaments, and assisting in improving pelvic problems such as relaxation and prolapse. The chasteberry extract is rich in anthocyanins, has antioxidant and protective effects, and can also assist in regulating hormone prolactin levels in the body, alleviate premenstrual syndrome and improve irregular menstrual cycles. The flaxseed lignan can bidirectionally regulate estrogen, make the growth and shedding of endometrium more regular, assist in improving the problems of excessive, insufficient, long or short menstruation, alleviate women's physiological discomfort while simulating the effect of estrogen to a certain extent to alleviate menopausal symptoms and prevent significant decline in ovarian function. Therefore, the ovarian care formula can directly act on ovaries, with fast results and no side effects.

### DETAILED DESCRIPTION OF THE EMBODIMENTS

Next, the technical solution in embodiments of the present disclosure will be clearly and completely described, obviously, the described embodiments are only some embodiments of the present disclosure, but not all the embodiments. Based on the embodiments in the present disclosure, other embodiments obtained by persons of ordinary skill in the art without creative efforts are all included within the protection of scope of the present disclosure.

The present disclosure provides the following technical solution: an ovarian care formula containing A-SDGPLUS is prepared from the following raw materials in percentage by mass:
30%-50% of postbiotics, 15%-25% of chasteberry extract and 5%-10% of flaxseed lignan.

A preparation method for an ovarian care formula containing A-SDGPLUS comprises the following steps:
mixing postbiotics, a chasteberry extract and flaxseed lignan in percentage by mass described above, evenly stirring, and packaging to obtain an ovarian care solid drink.

The above prepared ovarian care solid drink has the effect of stimulating female hormone secretion while having the functions of promoting menstruation and nourishing the ovaries and uterus, repairing the pelvic floor, inducing collagen synthesis in both directions, enhancing immunity, delaying aging, maintaining youthful appearance, improving melasma, pigmentation, enlarged pores and acne skin, preventing breast atrophy and sagging, improving sexual vitality, treating and improving dysmenorrhea, emotional fluctuations, insomnia, lower back pain and hair loss.

The specific effects of the ovarian care formula are achieved as follows:

Specifically, a preparation method for the postbiotics comprises:
S101: activating condensed Weissman's bacteria to obtain an activating bacterial solution of condensed Weissman's bacteria;
S102: inoculating the activating bacterial solution of condensed Weissman's bacteria into a fermentation medium to be fermented to obtain a fermentation broth of condensed Weissman's bacteria; and
S103: centrifuging the fermentation broth of condensed Weissman's bacteria, and then freeze-drying separated supernatant to obtain postbiotics.

The above prepared postbiotics can promote the synthesis of human collagen synthesis, maintain an ovarian structure and support an ovarian function, prevent premature ovarian failure, regulate endocrine function and assist in maintaining normal secretion levels of estrogen, various health problems, caused by decreased estrogen levels, including menopausal symptoms and reproductive system aging, are prevented by maintaining stable water solubility of estrogen, while enhancing the elasticity and contractility of the pelvic fascia, urethral and bladder ligaments, and assisting in improving pelvic problems such as relaxation and prolapse.

Specifically, in step S102, the fermentation medium is prepared from the following raw materials in parts by mass:
1-15 parts of peptone, 15-35 parts of yeast extract, 5-10 parts of glucose, 1-15 parts of corn starch, 1-15 parts of dipotassium hydrogen phosphate, 0.1-1 part of magnesium sulfate, 0.01-0.1 part of manganese sulfate and 500-1200 parts of distilled water.

Specifically, in step S102, the fermentation temperature of the activating bacterial solution of the condensed Weissma's bacteria during the fermentation is 30-50° C., the speed of a table concentrator is 100-500 rpm, and the fermentation time is 15-30 h.

Specifically, a preparation method for the chasteberry extract comprises:
S201: taking 500-600 parts of dried chasteberry raw material, washing, smashing, boiling, and filtering floating impurities;
S202: after repeatedly boiling three times, adding 1-10 parts of carrier maltodextrin powder dusting through a spray tower, and filtering via a 100-mesh sieve to obtain the chasteberry extract.

The above prepared chasteberry extract is rich in anthocyanins, has antioxidant and protective effects, and can also assist in regulating hormone prolactin levels in the body, alleviate premenstrual syndrome, and improve irregular menstrual cycles.

Specifically, a preparation method for the flaxseed lignan comprises:
S301: smashing flaxseed fat dregs, filtering via a 10-80 meshed sieve, adding a 70-99.9% ethanol solution and alkali, performing reflux extraction, and then filtering an extraction solution;
S302: concentrating the extraction solution to a proportion of 1.05-1.1, and adjusting pH to 6.5-7.5 with an acid;
S303: loading the concentrated solution onto a macroporous adsorbent resin, performing primary elution with water once and then performing secondary elution with 20-50% ethanol solution, concentrating and drying the concentrated solution to obtain 50-70% flaxseed lignan; and
S304: dissolving the dried flaxseed lignan crude product with a fat-soluble solvent, filtering and drying a filtrate to obtain ≥98% flaxseed lignan named A-SDGPlus, i.e., flaxseed lignan rich in ovarian care components.

The above prepared flaxseed lignan can bidirectionally regulate estrogen, make the growth and shedding of endometrium more regular, assist in improving the problems of excessive, insufficient, long or short menstruation, alleviate women's physiological discomfort while simulating the effect of estrogen to a certain extent to alleviate menopausal symptoms and prevent significant decline in ovarian function.

Specifically, in step S301, an addition ratio of ethanol solution to flaxseed fat dregs is 1:5-1:25 by weight, and an addition ratio of alkali to flaxseed fat dregs is 1-4% by weight.

Specifically, in step S301, the reflux extraction temperature is 70-80° C., and the extraction time is 3-4 h.

Although the embodiments of the present disclosure have been illustrated and described, it is understood that multiple changes, modifications, replacements and deformations can be made to these embodiments by persons of ordinary skill in the art without departing from the principle and spirit of the present disclosure, and the scope of the present disclosure is limited by appended claims and equivalents thereof.

What is claimed is:

1. An ovarian care formula containing A-SDGPLUS, which is prepared from the following raw materials in percentage by mass:
30%-50% of postbiotics, 15%-25% of chasteberry extract and 5%-10% of flaxseed lignan.

2. A preparation method for the ovarian care formula containing A-SDGPLUS according to claim 1, comprising the following steps:
mixing postbiotics, a chasteberry extract and flaxseed lignan in percentage by mass described above, evenly stirring, and packaging to obtain an ovarian care solid drink.

3. The preparation method for the ovarian care formula containing A-SDGPLUS according to claim 2, wherein a preparation method for the postbiotics comprises:
S101: activating condensed Weissman's bacteria to obtain an activating bacterial solution of condensed Weissman's bacteria;
S102: inoculating the activating bacterial solution of condensed Weissman's bacteria into a fermentation medium to be fermented to obtain a fermentation broth of condensed Weissman's bacteria; and
S103: centrifuging the fermentation broth of condensed Weissman's bacteria, and then freeze-drying separated supernatant to obtain postbiotics.

4. The preparation method for the ovarian care formula containing A-SDGPLUS according to claim 3, wherein in the step S102, the fermentation medium is prepared from the following raw materials in parts by mass:
1-15 parts of peptone, 15-35 parts of yeast extract, 5-10 parts of glucose, 1-15 parts of corn starch, 1-15 parts of dipotassium hydrogen phosphate, 0.1-1 part of magnesium sulfate, 0.01-0.1 part of manganese sulfate and 500-1200 parts of distilled water.

5. The preparation method for the ovarian care formula containing A-SDGPLUS according to claim 3, wherein in the step S102, the fermentation temperature of the activating bacterial solution of condensed Weissman's bacteria during the fermentation is 30-50° C., the speed of a table concentrator is 100-500 rpm, and the fermentation time is 15-30 h.

6. The preparation method for the ovarian care formula containing A-SDGPLUS according to claim 2, wherein a preparation method for the chasteberry extract comprises:
S201: taking 500-600 parts of dried chasteberry raw material, washing, smashing, boiling, and filtering floating impurities; and
S202: after repeatedly boiling three times, adding 1-10 parts of carrier maltodextrin powder dusting through a spray tower, and filtering via a 100-mesh sieve to obtain the chasteberry extract.

7. The preparation method for the ovarian care formula containing A-SDGPLUS according to claim 2, wherein a preparation method for the flaxseed lignan comprises:
S301: smashing flaxseed fat dregs, filtering via a 10-80 meshed sieve, adding a 70-99.9% ethanol solution and alkali, performing reflux extraction, and then filtering an extraction solution;
S302: concentrating the extraction solution to a proportion of 1.05-1.1, and adjusting pH to 6.5-7.5 by using an acid;
S303: loading the concentrated solution onto a macroporous adsorbent resin, performing primary elution with water once and then performing secondary elution with 20-50% ethanol solution, concentrating and drying the concentrated solution to obtain 50-70% flaxseed lignan; and
S304: dissolving the dried flaxseed lignan crude product with a fat-soluble solvent, filtering and drying a filtrate to obtain ≥98% flaxseed lignan named A-SDGPlus, i.e., flaxseed lignan rich in ovarian care components.

8. The preparation method for the ovarian care formula containing A-SDGPLUS according to claim 7, wherein in the step S301, an addition ratio of ethanol solution to flaxseed fat dregs is 1:5-1:25 by weight, and an addition ratio of alkali to flaxseed fat dregs is 1-4% by weight.

9. The preparation method for the ovarian care formula containing A-SDGPLUS according to claim 7, wherein in the step S301, the reflux extraction temperature is 70-80° C., and the extraction time is 3-4 h.
